(19) United States
(12) Patent Application Publication
(10) Pub. No.: US 2002/0013579 A1
(43) Pub. Date: Jan. 31, 2002

(54) ROTATING ELECTROSURGICAL BLADE FOR CORNEAL RESHAPING

(76) Inventor: THOMAS A. SILVESTRINI, ALAMO, CA (US)

Correspondence Address: CHRIS JAMES, KERA VISION, INC., 48630 MILMONT DR., FREMONT,, CA, 94538-7353, US

(21) Appl. No.: 08/943,581

(22) Filed: Oct. 3, 1997

(51) Int. Cl. A61B 18/14

(52) U.S. Cl. 606/32; 606/41

(57) ABSTRACT

A rotatable electrosurgical apparatus for reprofiling a cornea is described. The apparatus includes one or more electrosurgical electrodes that extend radially outward from a center point. The electrodes are shaped to reform at least a portion of an anterior surface of the cornea. The electrodes are disposed on an electrode support, which is rotatable. The electrodes project from the bottom of a rotary handle, which rotates the electrodes about a central visual axis of the cornea. The rotary handle has a hollow bore and a viewing port. The apparatus includes a support base having a base ring for positioning on the eye. The base ring can hold a solution against the eye to even out irregularities in the cornea. The apparatus may include pads to exert pressure on the cornea to cause the cornea to bulge in desired areas. These bulged areas are more easily modified by the electrodes when energized. The pressure pads take different forms depending upon whether they are used for the correction of myopia, hyperopia or astigmatism.

### FIELD OF INVENTION

The present invention relates to the field of correcting refractive errors of the eye, and more particularly to corneal electrosurgery.

### DESCRIPTION OF THE RELATED ART

Anomalies in the overall shape of the eye can cause visual disorders. Hyperopia (“farsightedness”) occurs when the front-to-back distance in the eyeball is too short. In such a case, parallel rays originating greater than 20 feet from the eye focus behind the retina. In contrast, when the front-to-back distance of the eyeball is too long, myopia (“nearsightedness”) occurs and the focus of parallel rays entering the eye occurs in front of the retina. Astigmatism is a condition which occurs when the parallel rays of light do not focus to a single point within the eye, but rather have a variable focus due to the fact that the cornea refracts light in a different meridian at different distances. Some degree of astigmatism is normal, but where it is pronounced, the astigmatism must be corrected. Hyperopia, myopia, and astigmatism are usually corrected by glasses or contact lenses.

Another method for correcting those disorders is by reshaping the corneal surface through an operative procedure. Such methods include radial keratotomy (see, e.g., U.S. Pat. Nos. 4,815,463 and 4,688,570) and laser corneal ablation (see, e.g., U.S. Pat. No. 4,941,093). Other surgical techniques involve scraping or cutting the exterior corneal surface. Lieberman (U.S. Pat. No. 4,807,623) employs a pair of angled cutting blades that are rotated around the corneal center to excise an annular wedge from the cornea to correct refractive errors. Kilmer, et al. (U.S. Pat. No. 5,318,044) provides curved rotating blades that scrape the corneal surface to correct refractive errors. That patent is incorporated by reference herein.

Some other corneal reshaping techniques do not involve surgery, but rather apply a radio frequency electrical signal to remove corneal tissue noninvasively. One technique, conductive keratoplasty, described in U.S. Pat. No. 5,533,999, issued to Hood, et al., applies an RF current directly to symmetrical spots on the cornea. This technique heats the corneal tissue to shrink and steepen the tissue in order to correct hyperopia and astigmatism. Similarly, both Doss, et al. (U.S. Pat. No. 4,326,529) and Doss (U.S. Pat. No. 4,381,007) employ an electrode that is placed near but not physically touching the anterior corneal surface. An electrically conductive coolant is placed over the corneal surface and circulated around the electrode as RF energy is applied through the electrode. The RF apparently heats various stroma within the cornea and thereby reshapes it as a biological response to the heat generated by the RF.

Two related patents, Dobrogowski, et al. (U.S. Pat. No. 5,025,811) and Latina, et al. (U.S. Pat. No. 5,174,304) illustrate noninvasive methods for focal transcleral destruction of living human eye tissue. In general, these devices and their underlying procedures involve the use of electric currents for ablating eye tissue, particularly the ciliary process. No mention of corneal reshaping is made. These references also relate to the application of a DC signal to the eye employing an ionic solution within the electrosurgical probe. The use of RF is not disclosed. Further, the ablation process is performed by repeatedly applying the probe to 10-30 spots around the circumference of the eye.

### SUMMARY OF THE INVENTION

The present invention provides a rotatable electrosurgical apparatus for reprofiling a cornea. The apparatus includes one or more electrosurgical electrodes that extend radially outward from a center point. The electrodes are shaped to reform at least a portion of an anterior surface of the cornea. The electrodes are disposed on an electrode support, which is rotatable. The electrodes project from the bottom of a rotary handle, which rotates the electrodes about a central visual axis of the cornea. The rotary handle has a hollow bore and a viewing port. The apparatus includes a support base having a base ring for positioning on the eye. The base ring can hold a solution against the eye to even out irregularities in the cornea.

The apparatus may include pads to exert pressure on the cornea to cause the cornea to bulge in desired areas. These bulged areas are more easily modified by the electrodes when energized. The pressure pads take different forms depending upon whether they are used for the correction of myopia, hyperopia or astigmatism.

### DETAILED DESCRIPTION OF THE INVENTION

The present invention provides a rotary electrosurgical blade assembly for corneal reshaping and a related method. In the following description, numerous details are set forth in order to enable a thorough understanding of the present invention. However, it will be understood by those of ordinary skill in the art that these specific details are not required in order to practice the invention. Further, well-known elements, devices, process steps and the like are not set forth in detail in order to avoid obscuring the present invention.

Prior to explaining the details of the inventive procedures and devices, a short explanation of the physiology of the eye is provided.

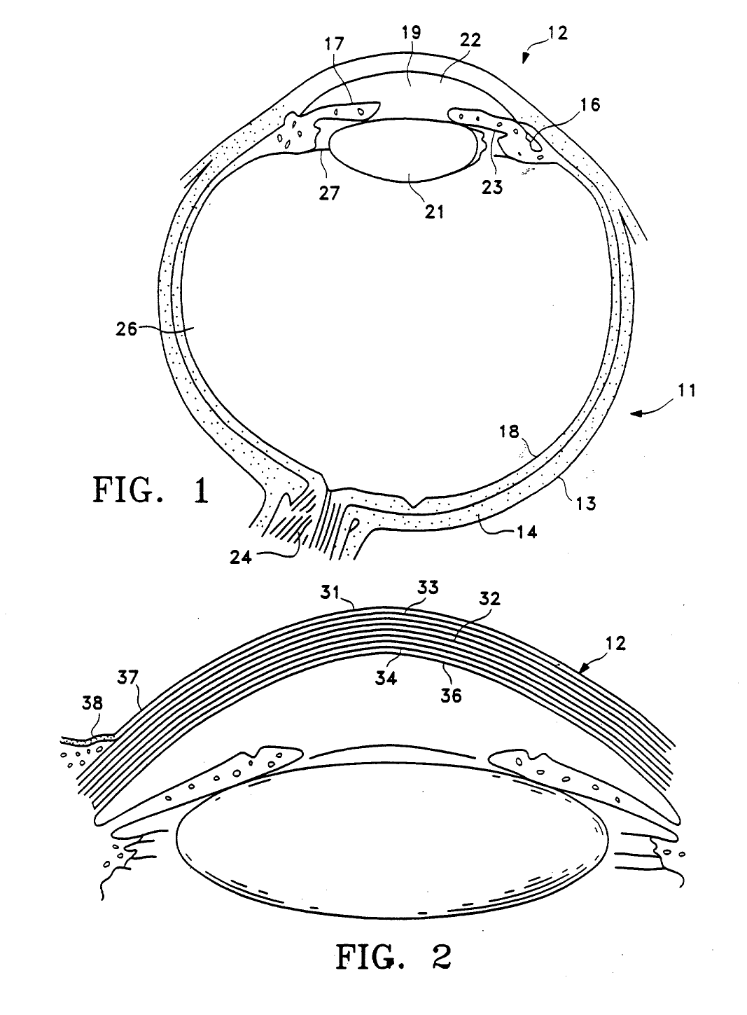

FIG. 1 shows a horizontal cross-section of the eye with the globe 11 of the eye resembling a sphere with an anterior bulged spherical portion representing the cornea 12.

The globe 11 of the eye consists of three concentric coverings enclosing the various transparent media through which the light must pass before reaching the light-sensitive retina 18. The outermost covering is a fibrous protective portion, the posterior five-sixths of which is white and opaque and called the sclera 13, and sometimes referred to as the white of the eye where visible to the front. The anterior one-sixth of this outer layer is the transparent cornea 12.

A middle covering is mainly vascular and nutritive in function and is made up of the choroid, ciliary body 16, and iris 17. The choroid generally functions to maintain the retina 18. The ciliary body 16 is involved in suspending the lens 21 and accommodation of the lens. The iris 17 is the most anterior portion of the middle covering of the eye and is arranged in a frontal plane. It is a thin circular disc similar in function to the diaphragm of a camera, and is perforate near its center by a circular aperture called the pupil 19. The size of the pupil varies to regulate the amount of light which reaches the retina 18. The iris divides the space between the cornea 12 and the lens 21 into an anterior chamber 22 and the posterior chamber 23. The innermost portion of covering is the retina 18, consisting of nerve elements which form the true receptive portion for visual impressions.

The retina 18 is a part of the brain arising as an outgrowth from the fore-brain, with the optic nerve 24 serving as a fiber tract connecting the retina part of the brain with the fore-brain. A layer of rods and cones, lying just beneath a pigmented epithelium on the anterior wall of the retina serve as visual cells or photoreceptors which transform physical energy (light) into nerve impulses.

The vitreous body 26 is a transparent gelatinous mass which fills the posterior four-fifths of the globe 11. At its sides it supports the ciliary body 016 and the retina 18. A frontal saucer-shaped depression houses the lens.

The lens 21 of the eye is a transparent bi-convex body of crystalline appearance placed between the iris 17 and vitreous body 26. Its axial diameter varies markedly with accommodation. A ciliary zonule 27, consisting of transparent fibers passing between the ciliary body 16 and lens 21 serves to hold the lens 21 in position and enables the ciliary muscle to act on it.

Referring again to the cornea 12, this outermost fibrous transparent coating resembles a watch glass. Its curvature is somewhat greater than the rest of the globe and is ideally spherical in nature. However, often it is more curved in one meridian than another, giving rise to astigmatism. Most of the refraction of the eye takes place through the cornea.

FIG. 2 is a more detailed drawing of the anterior portion of the globe showing the various layers of the cornea 12 making up the epithelium 31. An anterior limiting lamella 33, referred to as Bowman's membrane or layer, is positioned between the epithelium 31 and the stroma 32 of the cornea. The term “corneal mass” refers to the various stroma 32 between Bowman's layer 33 and Descemet's membrane 34. The corneal stroma 32 are made up of lamellae having bands of fibrils parallel to each other and crossing the whole of the cornea. While most of the fibrous bands are parallel to the surface, some are oblique, especially anteriorly. A posterior limiting lamella 34 is referred to as Descemet's membrane. It is a strong membrane sharply defined from the stroma 32 and resistant to pathological processes of the cornea.

The endothelium 36 is the most posterior layer of the cornea and consists of a single layer of cells and function to maintain transparency of the cornea 12. These epithelial cells are rich in glycogen, enzymes and acetylcholine and their activity regulates the transport of water and electrolytes through the lamellae of the cornea 12. The limbus 37 is the transition zone between the conjunctiva 38 and sclera on the one hand and the cornea 12 on the other.

In general, there are two distinct electrosurgical delivery probe types: the monopolar probe and the bipolar probe. An in-between electrosurgical configuration applicable to this invention also exists and is known as sesquipolar. In each instance, some section of the human body is used to complete a circuit between one pole and the other. In the monopolar probe device, there is a single active contact which is inserted or otherwise contacted with the human body and it is the site at which some body activity, e.g., desiccation, ablation, necrosis, fulguration, or the like, takes place. To complete the circuit in a monopolar device, there must be another contact which is inactive and placed against the body in a location remote from the active contact. By “inactive” is meant that only an insignificant temperature rise occurs at that contact point. One such method of ensuring that the inactive electrode is in fact “inactive” is to make it quite large in area. This causes the current to spread over a large area for completion of the circuit.

A bipolar electrode typically has two equal-area active electrodes contained in the same electrode probe-handle structure. This symmetric bipolar electrode design produces a significant temperature rise at both electrodes.

In a monopolar or sesquipolar configuration, only one electrode has an area of tissue contact producing a significant temperature rise. Unlike the monopolar configuration, however, the sesquipolar return electrode is not so remote, thereby limiting current flow through the body to the nearby return electrode. The return electrode area in the sesquipolar configuration electrode is usually at least three times the area of the active electrode and produces little or no tissue effect. For ocular surgery, the sesquipolar return electrode may be located on a non-remote region of the body, such as on the sciera or on a shaved area at the back of the patient's head.

There are a variety of effects that may occur depending upon the electrosurgical mode desired. For instance, there are both high temperature and low temperature desiccation effects when the active electrosurgical probe contact(s) are used to promote tissue desiccation. The resistance of the tissue in contact with the active probe electrode obviously varies with the tissue temperature and water content. A low temperature desiccation effect involves heating such that the temperature-time product causes tissue necrosis with little immediate denaturation or discoloration of the tissue. There is a transient decrease in local tissue impedance with little drying of tissue. In high temperature desiccation, there are significant increases in local tissue impedance and also in local tissue desiccation.

In the ablation mode, the electrosurgical energy density delivered largely causes the tissue near the probe contact to vaporize. The temperature at the electrode/tissue interface is increased significantly past the point of steam formation. The effect of electrical resistance varies during a specific radio frequency cycle and although there is sparking, carbonization is not usually significant and the effects of the device are relatively rapid.

Electrosurgical ablation and cutting produce an effect where a thin layer of tissue is vaporized (cutting) or where a larger section of tissue is vaporized (ablation). The line between “cutting” and “ablation” is not always clear.

Blended mode is essentially a combination of the cutting and coagulation (desiccation) modes. In blended mode, cutting or ablation with hemostasis is achieved.

The present invention employs electrosurgical ablation to reprofile the anterior surface of the cornea 301. For techniques that employ electrosurgery to modify the cornea from below the surface, please refer to U.S. patent application Ser. Nos. 08/194,207, 08/513,589, and 08/698,985, all of which are incorporated by reference herein.

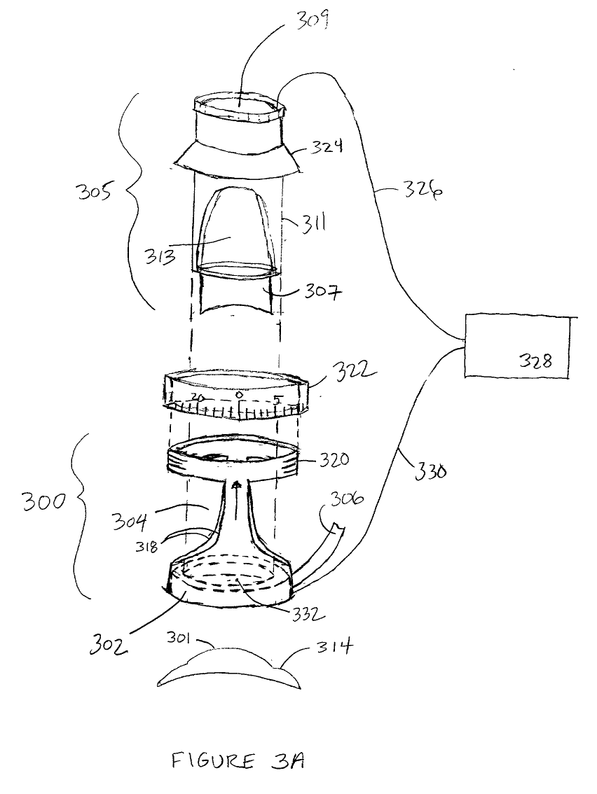

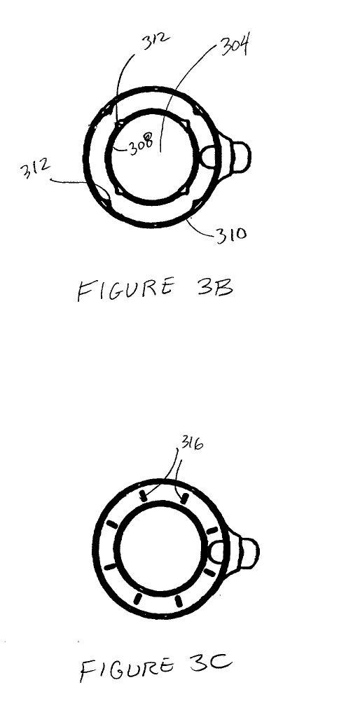

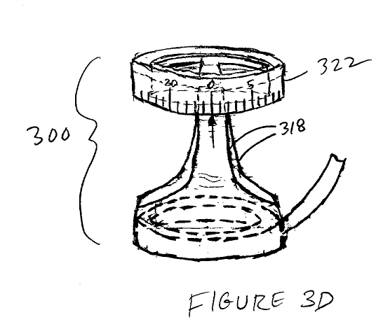

FIG. 3A illustrates a rotatable electrosurgical apparatus of the present invention, where the basic parts of the assembly are shown in an exploded view. In one embodiment, the components of the assembly include a generally cylindrical support base 300 having an annular base ring 302 and a cylindrical bore 304 extending through the support base. The base ring 302 may be implemented as a circumcorneal vacuum ring. A vacuum hose 306 connects the vacuum ring 302 to a vacuum pump (not shown). The vacuum ring 302 is configured so that it meets with and seals to the front of the eye, rendering the support base 300 relatively immobile when the support base 300 is applied to the front of the eye and a suitable vacuum is applied to the vacuum hose.

A rotary handle 305 having an electrosurgical blade assembly 307 is adapted for insertion into the support base 300. The rotary handle 305 has a hollow bore 309 to allow viewing of the corneal surface during operation of the apparatus. The side of the bore is also open to provide a viewing port 313. The inner diameter of the handle bore 309 is at least large enough so that the surgeon can see the blade assembly 307 by looking down into the bore 309. The bore 309 is desirably a length such that the ratio of the bore's length to its diameter is between 0.25:1 and 15:1; specifically between 0.4:1 and 1:1, at least about 1:1 and less than about 3:1; or at least 3:1 up to about 15:1. Preferably, the ratio is about 2.5:1. This sizing allows easy manipulation by the surgeon.

FIG. 3B illustrates a bottom view of the base ring 302 to show its internal structure when implemented as a vacuum ring. The vacuum ring 302 comprises an inner wall 308 having an inner diameter that allows the outer diameter of the rotary handle tube 311 to fit into the base ring 302.

The outer vacuum ring wall 310 forms the outside of the base ring 302. Interior to the vacuum ring 302 may be one or more ridges 312 which extend down to the corneal surface when the support base 300 is attached to the eye. These ridges 312 may be made of conductive material, whereas the surrounding support base structure, such as the inner wall and outer wall, are made of insulative material. The ridges may be coupled to an electrosurgical generator. Using this configuration, the ridges may act as return electrodes when operating in sesquipolar mode. These return electrodes may be positioned to rest on the sclera 314 or translimbal region of the eye.

FIG. 3C shows an alternative arrangement of return electrodes comprising radial vanes 316 that extend downward through the vacuum ring 302 to make contact with the sclera 314 or translimbal region.

Similar vacuum ring configurations for other purposes are described in U.S. Pat. No. 5,403,335, issued to Loomas et al., and assigned to the assignee of the present invention. That patent is incorporated by reference herein.

Alternatively, the support base 300 can rest on the sclera 314 without use of a vacuum ring. In its place, a base ring 302 of resilient material can be used as a substitute for the hollow annular vacuum ring. As another alternative, the bottom of the base ring 302 can be serrated to hold the ring in place.

The support base 300 may include two standoffs 318, shown here as one behind the other on opposite sides of the support base 300. The standoffs 318 are topped by a support ring 320. The support ring 320 may have an inner diameter greater than or equal to that of the base ring 302. As shown in FIG. 3D the support ring 320 may be threaded and screwed into a calibrated micrometer-like adjustment ring 322, similar to that used in the Kilmer '044 patent. A collar 324 of the handle 305 rests on top of the adjustment ring 322. By rotating the adjustment ring 322, the adjustment ring 322 controls the axial depth of the blades 307.

Because only two thin standoffs 318 are employed to support the adjustment ring 322, the surgeon is provided with a relatively large viewing port area to allow observation of the operational steps taking place at the corneal surface. The base 300 may have substantially more open area than closed area to maximize visibility. As an alternative, the support base may not include the standoffs 318 and support ring 320. The base ring 302 alone may serve as a guide for the handle to increase viewing area. Further, the entire support base may be omitted when performing the surgical procedure. In that case, the surgeon essentially performs the operation “free hand.”

The electrode blade assembly 307 is coupled through one lead 326 to an electrosurgical generator 328 so as to act as an active electrode. In a sesquipolar configuration, the other lead 330 of the generator 328 may be coupled to return electrodes 312 or 316 disposed on the bottom of the support base 300, as shown in FIGS. 3B and 3C. The return electrodes 312 or 316 rest on the scleral portion 314 of the eye. Alternatively, in a monopolar configuration, the return electrode may be placed elsewhere on the patient's body.

When using the complete support base as a guide, the surgeon positions the support base 300 on the eye so that it is centered over the central visual axis of the cornea 301. A vacuum is applied to hold the base in place if the vacuum ring embodiment is employed. The surgeon inserts the rotary handle 305 into the support base 300 so that the collar 324 rests on the adjustment ring 322. The surgeon rotates the adjustment ring 322 so that the electrode blade assembly 307 contacts the cornea 301. Because the invention employs electrical energy, the blades 307 need only lightly touch the corneal surface.

Alternatively, the blades 307 may be positioned near the corneal surface without touching the surface when a conducting medium such as saline is present. For this purpose, the blades 307 may be placed within a range of approximately 50-500 microns from the eye. It is the electrical contact, not the mechanical contact, between the blades and the cornea that achieve modification of the corneal surface. Initial electrical contact may be indicated by a continuity tester, as is well known in the art. The proper distance to achieve local conduction between the blades and the cornea can instead be determined by the surgeon by energizing and slowly lowering the energized blade assembly 307 towards the cornea while viewing the effects on the corneal surface 301. To aid in blade placement, the distance from the cornea may be measured with a traveling scale, such as an electronic dial caliper manufactured by Mitsutoyo, Inc. The scale can be zeroed when the blades touch the cornea.

The surgeon energizes the rotary blade assembly 307 with an RF current from the generator 328 to achieve volume modification of the cornea 301. Preferably, the procedure should be performed while the eye is bathed in a solution, such as saline, in order to even out irregularities in the tissue caused by uneven hydration of corneal tissue. The solution is held in the bore 332 of the base ring 302, and does not leak because of the tight fit between the base ring 302 and the eye.

The current employed by the present invention to achieve volume modification is typically a radio frequency current approximately on the order of 500 KHz or more. Additionally, the RF energy is often delivered in a pulsed or a continuous, non-pulsed operation depending on the exact effects desired. For further information concerning the electrical characteristics of electrosurgical waveforms, and electrosurgery in general, please refer to J. A. Pearce, Electrosurgery, John Wiley & Sons, 1986; U.S. Pat. No. 4,438,766 issued to Bowers; the SSE2K Electrosurgical Generator Service and Instruction Manuals (1982, 1980), the SSE2L Electrosurgical Generator Instruction Manual (1991), and the Force 2 Electrosurgical Generator Instruction Manual (1993), Valleylab. All of these references are incorporated by reference herein.

The rotary blades 307 may be energized by a common electrosurgical generator such as the Force 2, manufactured by Valleylab, Inc. The generator 328 includes settings for providing the appropriate electrosurgical waveforms for cutting, coagulation or blended modes. The wave shape for each mode is specified in the Valleylab generator manual. Cutting or ablation is performed with a 510 KHz continuous sinusoid. Coagulation (desiccation) employs a 510 KHz damped sinusoidal burst with a repetition frequency of 31 KHz. In blended modes, the generator outputs a 510 KHz sinusoidal burst at various duty cycles recurring at 31KHz. Those skilled in the art will recognize that the present invention is not limited to the generators, particular wave shapes or electrical characteristics disclosed herein.

The blades 307 initially may be energized at a low power setting (e.g., 0-5 watts) for approximately 1-5 seconds or longer. During energization of the blades, the surgeon rotates the blade assembly 307 and observes the volume reduction process to ensure that tissue is being safely removed or shrunk from the proper corneal regions. Typically, this observation may be performed through an ophthalmic microscope commonly used in opthalmological surgical procedures. The observation is conducted through the viewing ports or by removing the entire apparatus after each iteration of the procedure.

After completion of the corneal volume reduction step, the support base 300 and rotary handle assembly 305 are removed and the curvature of the corneal surface is then measured. One common method for measuring corneal curvature employs the Placido ring technique embodied in the Corneal Topography System manufactured by Eyesys of Houston, Tex. Curvature may also be measured using the technique described in allowed U.S. patent application Ser. No. 08/200,241, assigned to the assignee of the present invention, and incorporated by reference herein. The procedure may be repeated if insufficient correction has occurred. When repeating the procedure, the surgeon may increase the output power to reduce a greater volume of tissue until the desired effect is achieved. The surgeon may also lower the blades 307 by adjusting the adjustment ring 322.

FIGS. 4-10 illustrate side and bottom views of various configurations of the rotary blade assembly. FIG. 4 illustrates an embodiment of a single blade assembly for correction of myopia. A single active blade electrode 400 is disposed on an insulating electrode blade support 401 and extends radially outward from a center point 402. The broken lines of the bottom views of FIGS. 4-10 illustrate the full circles that can be swept by the blades and blade supports of those figures. In FIG. 4, the electrode is shaped to flatten the central portion of the anterior surface of the cornea 301. By rotating the electrode 400 in ablation mode, a surgeon may modify the volume of the central corneal region in order to correct myopia.

Selecting the proper blade shape for the desired correction is relatively easy using well-known relationships between the radius of corneal curvature and refractivity. The patient is given an eye exam to determine the degree of correction necessary. The refractive power correction is then correlated to a desired radius of corneal curvature, as is known in the art. A blade, such as that of FIG. 4, is chosen with this radius to reform the cornea to the correct radius. Blade selection may be refmed by conforming the blade shape to the shape determined by known topographical techniques as necessary for proper correction.

FIG. 5 illustrates a single blade embodiment for the correction of hyperopia. An active electrode 500 is disposed on an insulating blade support 501 and extends radially outward from a center point 502. The active electrode 500 is disposed near the periphery of the rotary blade assembly 307. When the blade 500 is rotated by the surgeon in ablation mode, the blade removes an annulus of corneal tissue in order to steepen the central corneal region so as to correct hyperopia.

Generally, the blade electrode of FIG. 4 is rotated 360 degrees to correct myopia. Similarly, the blade electrode 500 of FIG. 5 is rotated 360 degrees to correct hyperopia. Those skilled in the art will recognize that the blades can be rotated over smaller angular sectors in order to vary the correction of refractive error. For example, the blades of any of the embodiments described herein may be rotated through various angular sectors to correct astigmatism.

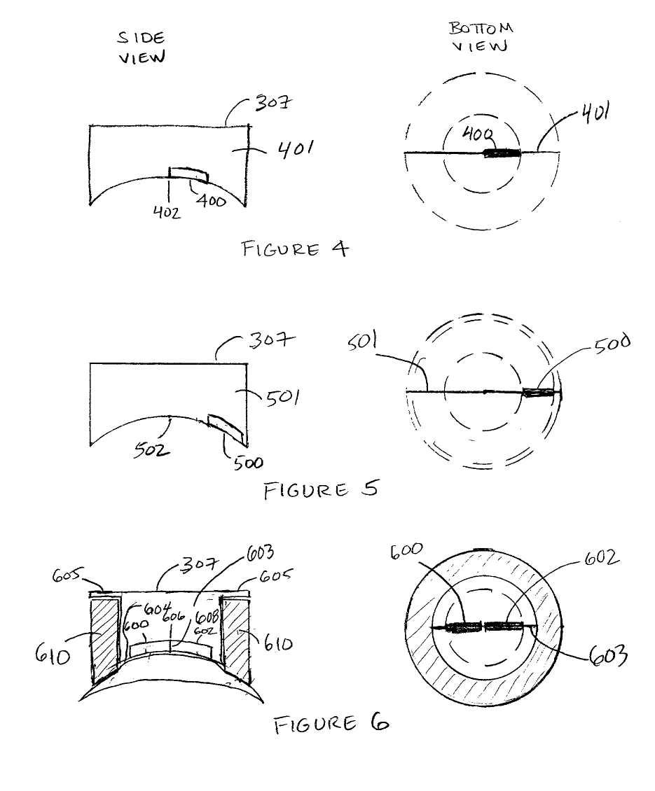

FIG. 6 illustrates side and bottom views of a dual blade embodiment of the blade assembly 307 for correcting myopia. The assembly 307 includes two active electrodes 600 and 602 disposed on an insulating blade support 603 along a curved diameter line 604 passing through a center point 606. Each of the blade electrodes 600 and 602 is curved to reform the shape of the central corneal region to correct myopia. The blade electrodes 600 and 602 may be separated by an insulator 608. The blades 600 and 602 may be electrically coupled together by a wire (not shown) in the rotary handle. The wire itself is connected to the active lead of the generator. Alternatively, one integrated conducting blade electrode (not shown) that is symmetric about the center point may replace the two separate electrodes 600 and 602.

The blade assembly 307 may also fit into an annular peripheral pressure pad 610, which is shown in cross-section in the side view of FIG. 6. The insulative pad is placed inside the bore 332 of the base ring 302, and allowed to move freely in the axial direction. The pad 610 may include a vertical groove on its outer side to accept a pin (not shown) in the base 302 so that the pad is fixed in the direction of rotation, but still allowed to move in the axial direction. Alternatively, the pad 610 may be mounted to the interior of the tube 311. The pad may rotate with the tube 311 or loosely placed in the tube 311 so that it is held in place on the eye while the tube 311 rotates. When the peripheral pad 610 is applied to the peripheral area on or near the cornea, the central corneal region bulges to provide a more well-defined region for ablation. Those skilled in the art will recognize that the peripheral pad may be employed with any of the blade assemblies described herein for modifying tissue near the center of the cornea.

The blade support 603 is mounted to the interior of the tube 311 of the rotary handle 305, for example, by thin brackets 605, so that the blade support 603 (and the blades 600 and 602) rotates as the handle 305 is rotated. (Generally, all blade assemblies described herein are mounted to the interior of the tube 311.)

The brackets 605 act as a stop to prevent upward movement of the pad 610. Thus, by using pads of different heights, the relationship between the bottom of the pad 610 and the edge of the blades 600 and 602 may be adjusted. This, in turn, adjusts the size of the corneal bulge when the assembly is placed on the eye, thereby giving a different resulting corneal curvature for the same blade. That is, the higher the bulge, the deeper the resulting tissue modification.

This dual blade configuration allows the surgeon to ablate a 360 degree region by rotating the assembly 307 through only 180 degrees because each blade ablates half of the total 360 degree region. Similarly, the blade assembly can be reproduced and orthogonally combined so that the electrodes are separated by 90 degrees. Further combinations can be made for smaller angular separations. Those skilled in the art will recognize that any of the blade assemblies 307 disclosed herein may be combined in this manner.

To effectively achieve multiplexing, each blade can also be independently energized to provide a higher current density per blade for the same amount of power. For example, the surgeon can rotate the dual blade assembly in one direction with only one blade energized, and then rotate the assembly back in the other direction with only the other blade energized.

FIG. 7 illustrates a dual blade assembly 307 for correcting hyperopia. Blades 700 and 702 are disposed on an insulating blade support 703 along a diameter line 704 passing through a center point 706. The blade electrodes 700 and 702 may be electrically coupled together in the same manner as in FIG. 6. The blade assembly may also include an insulative central pressure pad 708. The pad extends slightly below, about 0.1 mm, the portion of the blade support 703 adjacent the pad 708. The blade support 703 is mounted on the rotary handle 305 so that the blade support (and the blades) rotate as the handle is rotated. The pad 708 is rotatably coupled to the blade support so that when the blade assembly 307 is applied to the eye, the pad 708 is held stationary against the cornea 301 by friction as the blade support 703 swivels around the pad 708 when the handle 305 is rotated. Alternatively the pad 708 may be fixed to the handle 305. When the pad 708 is applied to the central area of the cornea, the peripheral corneal surface bulges to provide a more well-defined region for ablation. The size of the bulge is governed by the relative distance between the bottom of the pad 708 and the edge of the blades 700 and 702. Those skilled in the art will recognize that a central pressure pad may be employed in any of the blade assemblies described herein for modifying tissue outside the center area of the cornea.

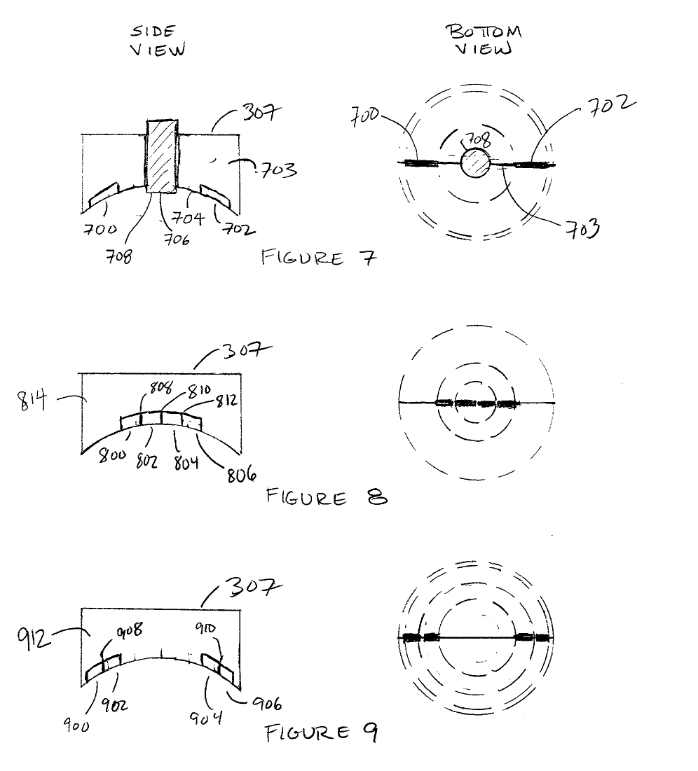

FIG. 8 illustrates another embodiment of the dual blade myopic correction assembly 307. In this embodiment, the active electrode assembly is divided into four active electrodes 800, 802, 804 and 806. The electrodes are separated by insulative portions 808, 810 and 812, respectively, of a blade support 814. The electrodes 802 and 804 may be electrically coupled to each other to form a first set of coupled electrodes, and electrodes 800 and 806 may be electrically coupled together to form a second set of coupled electrodes. The four electrodes of this embodiment are configured to have effectively the same blade area for contact with the cornea as the two electrodes of the embodiment of FIG. 6.

By employing this configuration, the sets of electrodes can be energized independently of each other using a simple switching circuit between the generator and the blades. For example, the surgeon can ablate the central corneal region with the first set of coupled electrodes through a given angular sector using a given axial pressure and power setting. Then, the surgeon can ablate a concentric region with the second set of coupled electrodes through the same or another angular sector using the same or a different axial pressure and the same or different power. In this manner, the surgical procedure is effectively multiplexed.

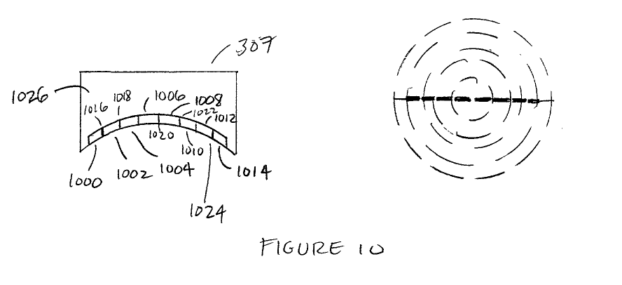

FIG. 9 illustrates another embodiment of the dual blade assembly for hyperopic correction. This embodiment features four blades 900, 902, 904 and 906 mounted on an insulating blade support 912. The blades 902 and 904 may be electrically coupled to form a first coupled set of electrodes, and electrodes 900 and 906 may be electrically coupled to each other to form a second set of coupled electrodes. Electrodes 900 and 902 are separated by an insulative portion 908 of the blade support 912. Electrodes 904 and 906 are separated by an insulative portion 910. These blades may be operated by the surgeon in a manner similar to that described with respect to FIG. 8, and may include a central pressure pad (not shown) such as that illustrated in FIG. 8.

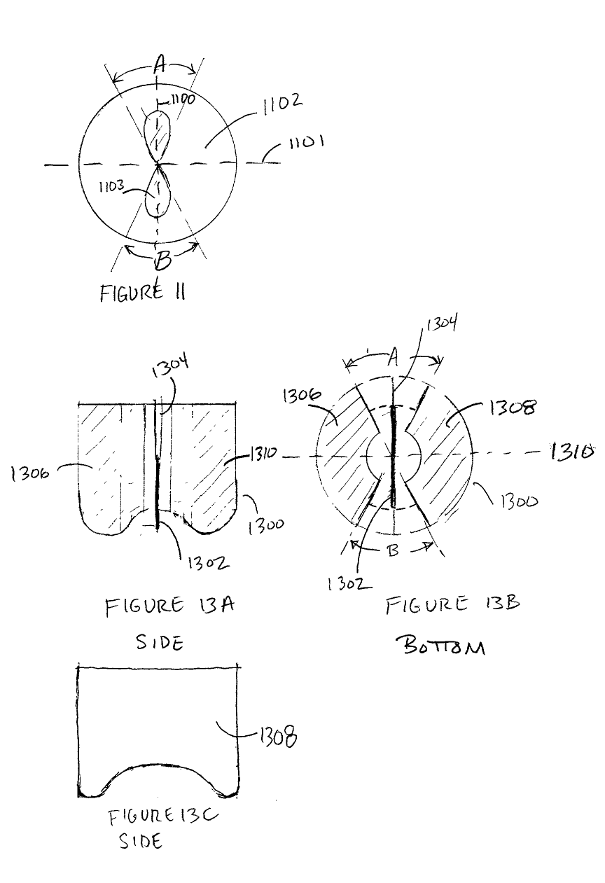

FIG. 10 illustrates a combination electrode blade assembly 307. This embodiment includes eight blade electrodes 1000, 1002, 1004, 1006, 1008, 1010, 1012, and 1014, separated by insulative portions 1016, 1018, 1020, 1022 and 1024, respectively, disposed on a blade support 1026. These electrodes may be electrically coupled in any manner and energized in any sequence to correct myopia, hyperopia, astigmatism or any other error correction desired by the surgeon.

As mentioned above, the present invention may be employed to treat astigmatism. Referring to FIG. 11, astigmatism occurs, generally, when the curvature of the anterior surface is not uniform along the circumference of the cornea, resulting in a steep axis 1100 and a flat axis 1101 along perpendicular meridians. The steeper axis is known as the axis of astigmatism 1100. A butterfly or figure-8-shaped region 1103 about the astigmatic axis 1100 is steeper than the surrounding region 1102 of the cornea. To correct astigmatism, the region 1103 must be flattened to cause the cornea to become reasonably symmetrical and more spherical in shape.

Blade assemblies such as those shown in FIGS. 6 and 8 may be employed to flatten the steepened region 1103 along the astigmatic axis 1100. Using those blade assemblies, a surgeon would not rotate the assemblies through a full 360 degree angle, but rather would only rotate them through angular sectors A and B to ablate the steepened tissue.

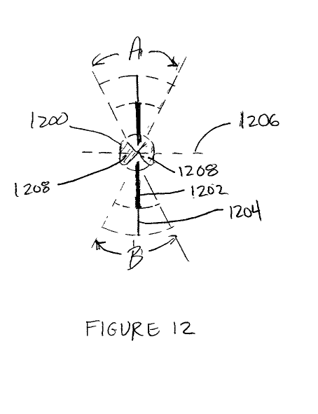

FIGS. 12-14 illustrate pressure pads that may be employed in the correction of astigmatism. The pads create bulges in the corneal regions adjacent the point of contact between the pads and the cornea. By making those regions more prominent, the pads make it easier for the surgeon to ensure that the correct areas of the cornea are modified.

FIG. 12 illustrates a bottom view of a central astigmatic pressure pad 1200 similar to the central pressure pad of FIG. 7 along with a blade 1202 and a blade support 1204. The pad 1200 is rotatably coupled to the blade support 1204. Unlike the pad of FIG. 7, this pad 1200 does not apply a uniform disc of pressure to the central corneal region. Instead, the pad has a butterfly shape to complement the steep butterfly region 1103 of the astigmatic cornea. The pad 1200 is applied to the flatter regions near the corneal center in order to cause the steep areas near the center to bulge. A first axis 1206 of the pad 1200 is applied to the flat corneal axis 1101. Wings 1208 of the pad limit rotation of the blade to the angular sectors A and B about the steep astigmatic axis 1100. The dashed lines indicate the limit angular sector swept by the blades 1202 and blade support 1204.

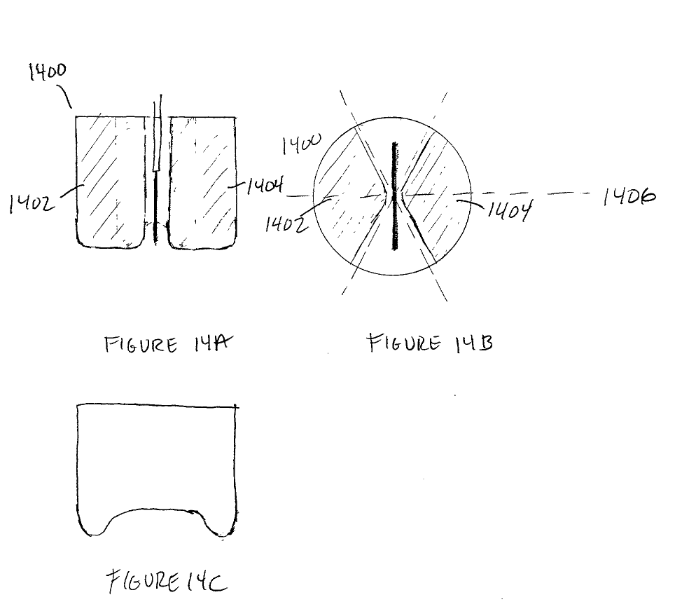

FIGS. 13A and 13B illustrate a side cross-sectional view and a bottom view, respectively, of an annular peripheral astigmatic pressure pad 1300 similar to the peripheral pad of FIG. 6, along with a blade 1302 and a blade support 1304. The pad 1300 is mounted to the base ring 302. However, unlike the pad of FIG. 6, this pad does not circumscribe a complete 360 degree annulus. Instead, the pad 1300 is shaped so that no pressure is applied to the angular sectors A and B, thereby causing those regions to bulge when pressure is applied. The pad comprises first and second annular segments or wings 1306 and 1308, respectively. FIG. 13C is a side view (not sectional) of FIG. 13A rotated 90 degrees to show the side of wing 1308. A first axis 1310 is disposed along the flat corneal axis 1101. The annular segments limit rotation of the blade 1302 to the angular sectors A and B about the steep astigmatic axis 1100. The pad 1300 also allows the blade to contact the center of the cornea.

FIGS. 14A and 14B illustrate a variation of FIGS. 13A and 13B, wherein pressure is applied not only to a peripheral annular region, but also to the central corneal region in which the corneal surface is relatively flat. The pad 1400 is mounted to the base ring 302. The pad 1400 comprises first and second wings 1402 and 1404, respectively. FIG. 14C is a side view (not sectional) of FIG. 14A rotated 90 degrees to show wing 1404. A first axis 1406 is disposed along the flat axis 1101. The wings apply pressure to both the central and peripheral corneal regions to limit rotation. As a result, the corneal surface bulges in the angular sectors A and B, almost as if a combination of the central astigmatic and peripheral astigmatic pressure pads were applied.

Of course, any of the pad configurations disclosed herein may be varied to cause different corneal regions to bulge.

As apparent from the discussion above, the present invention exhibits advantages over prior art mechanical techniques. Because the electrical blade assembly requires only light or no mechanical contact, the invention does not traumatize the corneal surface and provides a more controlled tissue removal procedure than mechanical methods. When a mechanical blade scrapes a cornea, tissue in the path of the advancing blade can bulge, leading to a possible gash in the bulge or other non-uniformity in the surface modification. Further, debris resulting from mechanical scraping in the path of the advancing blade can jam the blade, also leading to non-uniformities. In contrast, electrical ablation by the blade assembly of the present invention vaporizes tissue cleanly in the path of the blades.

While the invention has been described with reference to numerous specific details, one of ordinary skill in the art will recognize that the invention can be embodied in other specific forms without departing from the spirit of the invention. Further, all patents, applications and other references cited herein are incorporated by reference herein. One of ordinary skill in the art will understand that the invention is not to be limited by the foregoing illustrative details, but rather is to be defined by the appended claims.

What is claimed is:

1. A rotatable electrosurgical apparatus for reprofiling a cornea, the apparatus comprising:
at least one active electrosurgical electrode extending radially outward from a center point, wherein the at least one electrode is shaped to reform at least a portion of an anterior surface of the cornea; and
an electrode support on which the at least one electrode is disposed, wherein the electrode support is rotatable.

2. The apparatus of claim 1, further comprising a rotary handle for rotating the at least one electrode about a central visual axis of the cornea, wherein the at least one electrode projects from the bottom of the rotary handle.

3. The apparatus of claim 1, wherein the rotary handle comprises a hollow bore.

4. The apparatus of claim 1, further comprising a support base having a base ring for positioning on the eye, the support base including a viewing port.

5. The apparatus of claim 2, the handle including a viewing port.

6. The apparatus of claim 4, the base ring being adapted to hold a solution against the eye, wherein the solution evens out irregularities in the cornea.

7. The apparatus of claim 4, further comprising a return electrode projecting from the bottom of the base ring for contacting the eye.

8. The apparatus of claim 1, the at least one electrode including at least two active electrodes disposed along a diameter line passing through the center point.

9. The apparatus of claim 8, wherein the electrodes are adapted to be energized independently of each other.

10. The apparatus of claim 8, the diameter line having two radial portions, wherein at least two active electrodes are disposed along each radial portion.

11. The apparatus of claim 8, the diameter line having two radial portions, wherein at least one active electrode is disposed along each radial portion, and each electrode in a first radial portion is electrically coupled to a corresponding electrode in a second radial portion along the same diameter line to form at least one set of coupled electrodes.

12. The apparatus of claim 11, wherein at least two active electrodes are disposed along each radial portion, and the sets of coupled electrodes are adapted to be energized independently of each other.

13. The apparatus of claim 11, wherein the sets of coupled electrodes are adapted to be energized in a sequential manner.

14. The apparatus of claim 1, further comprising a central pressure pad disposed at the center point.

15. The apparatus of claim 1, further comprising an annular pressure pad.

16. A rotatable electrosurgical apparatus for reprofiling a cornea of an eye, the apparatus comprising:
a rotary handle; and
at least one active electrosurgical electrode projecting from the bottom of the rotary handle and extending radially outward from a center point, wherein the at least one electrode is shaped to reform at least a portion of an anterior surface of the cornea.

17. The apparatus of claim 16, further comprising a support base having a base ring for positioning on the eye, the support base including a viewing port.

18. The apparatus of claim 17, the handle including a viewing port.

19. The apparatus of claim 16, further comprising a base ring adapted for positioning on the eye, the base ring having a bore for receiving the rotary handle.

20. The apparatus of claim 19, the base ring being adapted to hold a solution against the eye, wherein the solution evens out irregularities in the cornea.

21. The apparatus of claim 19, further comprising a return electrode projecting from the bottom of the base ring for contacting the eye.

22. The apparatus of claim 16, the at least one electrode including at least two active electrodes disposed along a diameter line passing through the center point.

23. The apparatus of claim 22, wherein the electrodes are adapted to be energized independently of each other.

24. The apparatus of claim 22, the diameter line having two radial portions, wherein at least two active electrodes are disposed along each radial portion.

25. The apparatus of claim 22, the diameter line having two radial portions, wherein at least one active electrode is disposed along each radial portion, and each electrode in a first radial portion is electrically coupled to a corresponding electrode in a second radial portion along the same diameter line to form at least one set of coupled electrodes.

26. The apparatus of claim 25, wherein at least two active electrodes are disposed along each radial portion, and the sets of coupled electrodes are adapted to be energized independently of each other.

27. The apparatus of claim 25, wherein the sets of coupled electrodes are adapted to be energized in a sequential manner.

28. The apparatus of claim 16, further comprising a central pressure pad disposed at the center point.

29. The apparatus of claim 16, further comprising an annular pressure paid.

30. A corneal pressure pad for astigmatic correction comprising:
a first axis of pad material for applying pressure to an anterior surface of a cornea along a flat axis of the cornea; and
wings of pad material adapted to limit rotation of a blade to an angular region about a steep astigmatic axis of the cornea.

31. The pad of claim 30, wherein the wings are adapted for application to a central corneal region.

32. The pad of claim 30, wherein the wings are adapted for application to a peripheral corneal region.

33. The pad of claim 30, wherein the wings are adapted for application to both a central corneal region and a peripheral corneal region.

34. The pad of claim 30, wherein the blade is an electrosurgical electrode.

35. A method for reprofiling a cornea comprising the steps of:
providing a rotatable electrosurgical apparatus having at least one active electrosurgical electrode extending radially outward from a center point, wherein the at least one electrode is shaped to reform at least a portion of an anterior surface of the cornea;
positioning the center point over a central visual axis of the cornea;
energizing at least one active electrode; and
rotating the at least one electrode about the central visual axis.

36. The method of claim 35, further comprising the steps of:
positioning a support base centered over the central visual axis; and
inserting the apparatus through the support base.

37. The method of claim 36, wherein the support base has a bore, the method further comprising the step of:
adding a solution to the bore of the support base, wherein the solution is held against the eye to even out irregularities in the cornea.

38. The method of claim 35, the providing step comprising the step of providing a rotatable electrosurgical apparatus having at least two active electrodes disposed along a diameter line passing through the center point.

39. The method of claim 38, the energizing step including the step of energizing the electrodes independently of each other.

40. The method of claim 38, the diameter line having two radial portions, wherein at least two active electrodes are disposed along each radial portion.

41. The method of claim 38, the diameter line having two radial portions, wherein at least one active electrode is disposed along each radial portion, and each electrode in a first radial portion is electrically coupled to a corresponding electrode in a second radial portion along the same diameter line to form at least one set of coupled electrodes.

42. The method of claim 41, wherein at least two active electrodes are disposed along each radial portion, the energizing step comprising the step of energizing the sets of coupled electrodes independently of each other.

43. The method of claim 41, the energizing step comprising the step of energizing the sets of coupled electrodes in a sequential manner.

44. The method of claim 35, further comprising the step of applying pressure to a central corneal region.

45. The method of claim 35, further comprising the step of applying pressure to a peripheral corneal region.

46. The method of claim 35, further comprising the step of:
applying pressure to an anterior surface of the cornea along a flat axis of the cornea, wherein
the rotating step comprises the step of limiting rotation of the at least one electrode to an angular region about a steep astigmatic axis of the cornea.

47. The method of claim 46, wherein the applying step comprises the step of applying pressure to a central corneal region.

48. The method of claim 46, wherein the applying step comprises the step of applying pressure to a peripheral corneal region.

49. The method of claim 46, wherein the applying step comprises the step of applying pressure to both a central corneal region and a peripheral corneal region.
